(19) United States
(12) Patent Application Publication
(10) Pub. No.: US 2025/0009817 A1
(43) Pub. Date: Jan. 9, 2025

(54) IMMUNOMODULATING COMPOSITION CONTAINING GASSERI

(71) Applicant: THE CATHOLIC UNIVERSITY OF KOREA INDUSTRY-ACADEMIC COOPERATION FOUNDATION, Seoul (KR)

(72) Inventors: Mi-La CHO, Seoul (KR); Min-Jung PARK, Gwangmyeong-si (KR); Joo-Yeon JHUN, Yangcheon-gu (KR); Seung-Yoon LEE, Dongjak-gu (KR); Se-Gyeong HAN, Namwon-si (KR)

(73) Assignee: THE CATHOLIC UNIVERSITY OF KOREA INDUSTRY-ACADEMIC COOPERATION FOUNDATION, Seoul (KR)

(21) Appl. No.: 18/417,720

(22) Filed: Jan. 19, 2024

(30) Foreign Application Priority Data
Jan. 19, 2023 (KR) 10-2023-0007901
Jan. 18, 2024 (KR) 10-2024-0008205

(51) Int. Cl. A61K 35/747; A61K 31/436; A61K 35/00; A61P 37/04

(52) U.S. Cl. CPC A61K 35/747; A61K 31/436; A61P 37/04; A61K 2035/115

(57) ABSTRACT

Provided is an immunomodulatory treatment composition for a transplantation disease through combined administration of a microbiome and an immunosuppressant. According to the present disclosure, it was confirmed that in an allogeneic transplantation cell model, combined administration of FK506 and the microbiome suppressed alloresponse and regulated the activity of T cells and B cells. Further, it was confirmed that in an acute graft-versus-host disease animal model, the combined administration reduced the disease activity, increased the probability of survival, and protected the tissue damage. In addition, it was confirmed that the combined administration improved transplantation rejection diseases by reducing the activity of Th1 and Th17 and increasing the activity of Tregs, and thus can be usefully used as an immunomodulator for transplantation rejection.

### CROSS-REFERENCE TO RELATED APPLICATIONS

This application is based on and claims priority from Korean Patent Application No. 10-2023-0007901, filed on Jan. 19, 2023, with the Korean Intellectual Property Office, the disclosure of which is incorporated herein in its entirety by reference.

### TECHNICAL FIELD

The present disclosure relates to an immunomodulating composition comprising gasseri.

### BACKGROUND

An immune system needs to differentiate between self and non-self. When the immune system does not differentiate between the self and non-self, the immune system destroys cells and tissues of the body, and as a result, causes autoimmune diseases. Regulatory T cells actively suppress the activation of the immune system to prevent pathological self-reactivity and consequently to prevent autoimmune diseases. The regulatory T cells (Tregs) are a class of CD4+CD25+ T cells that suppress the activity of other immune cells. The immune system is well-equipped with a device capable of quickly identifying and rapidly destroying foreign species, pathogens, or inflamed tissue. The immune system has been a major barrier to tissue, organ, and cell transplantation as well as gene therapy. The major problems were generally chronic immunosuppression, encapsulation, and immune isolation. Unwanted side effects of chronic immunosuppression include increased susceptibility to opportunistic infections and tumor formation.

The mechanisms by which T cell responses to foreign (allogeneic or xenogeneic) proteins or cells or organs operate are significantly well known. Antigen presenting cells (APCs) are introduced into an inflammation or injury site (triggered by transplantation), and a peripheral T cell constantly monitors for the presence (allogeneic or xenogeneic) of foreign tissue or pathogens. Once these warning signals are recognized, APCs digest allogeneic or xenogeneic pathogens and are presented as antigen to a host immune system.

Transplantation refers to a process of taking a cell, a tissue, or an organ, that is, a graft, from one subject and transferring the graft to the other subject. A subject who provides the graft is called a donor, and a subject who receives the graft is called a recipient or host. In the case of the transplanted organ, rejection occurs due to an immune response to a histocompatible antigen (transplant antigen) on the cell surface of the graft. A case of long-term engraftment of the graft in a non-immunosuppressed recipient is limited to a case where the histocompatibility is completely or mostly identical, and a genetic relationship between the donor and the recipient is a factor that greatly determines an engraftment period of the graft. In general, the rejection rarely occurs in autografts and isografts, but the rejection occurs in almost all allografts. When transplanting tissues, organs, or the like, a genetic difference between the donor and the recipient is easily detected by a host immune system to cause a host-versus-graft response and/or a graft-versus-host response. This fact has been proven by rejection that occurs during tissue and organ transplantation (Nash et al., Blood, 80, 1838-1845, 1992). In addition, there have been reports that rejection of allotransplanted tissue occurs due to T cells activated by an immune response to MHC present on the surface of the transplanted cell.

Since immunosuppressants that non-specifically suppress T cells for inhibiting the transplantation rejection or transplantation rejection disease are generally accompanied with side effects, such as cytotoxicity, infection due to immunosuppression, diabetes, tremor, headache, diarrhea, hypertension, nausea, and renal dysfunction, there is a disadvantage that a long-term treatment effect is unlikely to be sustainable. In order to reduce serious side effects and increase the effect of immunosuppressive treatment, methods of co-administering or replacing immunosuppressants with different mechanisms of action have been attempted, especially in the field of organ transplantation, but combination or treatment of optimized co-administration of immunosuppressants is still incomplete. Therefore, there is an urgent need to develop new immunosuppressive or immunomodulatory therapies capable of reducing the side effects of conventional immunosuppressants and improving a therapeutic effect, and to discover new immunosuppressant candidates that are safer and have fewer side effects.

Meanwhile, immunosuppressants currently used are divided into corticosteroid, antimetabolite, calcineurin inhibitor, mammalian target of rapamycin inhibitor, antibody, etc., but these immunosuppressants exhibit an immunosuppressive effect by blocking the proliferation or activation of T cells of the immune system at different stages. T cells, the main target of immunosuppressants, are generated in the human thymus and mainly differentiate into type 1 helper cells (Th1) involved in cell-mediated immunity or type 2 helper cells (Th2) involved in humoral immunity. It has been known that the two T cell populations keep each other in check so as not to be overactivated, and when the balance is broken, abnormal reactions such as autoimmunity or hypersensitivity reactions occur.

In addition, new types of T cells such as immunoregulatory T cells (Tregs) and Th17 capable of regulating immune responses have been known. The Tregs may regulate Th1 cell activity, suppress the functions of abnormally activated immune cells, and regulate inflammatory responses. On the other hand, Th17 cells secrete IL-17, and maximize the signal of the inflammatory response to accelerate the progression of the disease. Recently, these Treg or Th17 have greatly emerged as new targets for immune disease treatment, and various immunomodulatory therapeutic agents are being studied (Wood, K. J. et al., Nat. Rev. Immunol. 12(6):417-430, 2012; Miossec, P. et al., Nat. Rev. Drug Discov. 11(10):763-776, 2012; Noack, M. et al., Autoimmun. Rev. 13(6):668-677, 2014).

Since conventional immunosuppressants that non-specifically suppress T cells are generally accompanied with side effects, such as cytotoxicity, infection due to decline in immunity, diabetes, tremor, headache, diarrhea, hypertension, nausea, and renal dysfunction, there is a disadvantage that a long-term treatment effect is unlikely to be sustainable. In order to reduce serious side effects and increase the effect of immunosuppressive treatment, methods of combining or replacing immunosuppressants with different mechanisms of action have been attempted, especially in the field of organ transplantation, but treatment using an optimized single compound is still incomplete.

Meanwhile, a microbiome refers to microorganisms that live in the human body and includes intestinal microorganisms. The number of microbiomes is at least twice greater than that of pure human cells, and the number of genes is at least 100 times greater therethan. Accordingly, since it is impossible to discuss human genes without mentioning microorganisms, the microbiome is also called the second genome. The microbiome is a field that can be widely used in the development of new drugs and research on treatments for incurable diseases by analyzing the principles of formation of beneficial and harmful bacteria, the relation between diseases, etc. In addition, the microbiome is used to develop foods, cosmetics, and treatments.

As Next Generation Sequencing (NGS) technology advances, the importance of intestinal microorganisms is emerging in human physiology and immune regulation, and thus the importance of probiotics capable of directly regulating the structure of a human intestinal microbiome is emerging. Recently, through many studies, as probiotics are known to be closely related to the human microbiome, the probiotics have attracted attention for their functions as regulators that change an intestinal environment. The intake of probiotics is known to not only promote digestion through the microbiome regulation function, but also suppress inflammatory bowel disease, infectious diseases, or harmful bacteria. In particular, it has been found that the probiotics improve the host immune system to have an effect on various immune-related diseases including atopy, rheumatism, and the like and cancers.

In addition, as the probiotics field grows significantly, much attention has also focused on prebiotics, synbiotics, and postbiotics. The prebiotics are defined as ‘substances that are selectively used by beneficial bacteria that contribute to health among the microorganisms in the host’, and representative examples thereof include dietary fiber and oligosaccharides that serve as food for lactic acid bacteria. The synbiotics are a form that contains both probiotics and prebiotics. Meanwhile, the postbiotics, which have recently been attracting attention, are materials that contain useful metabolites produced by probiotics and components of microorganisms and are clearly defined as ‘a non-living form of microorganisms beneficial to the health of the host or a formulation containing components of those microorganisms.’ The postbiotics are attracting attention as a new alternative material that can overcome the limitations of safety, functionality, and stability of conventional probiotic materials.

### SUMMARY

The present disclosure has been made in an effort to provide a pharmaceutical composition for prevention or treatment of transplantation rejection or transplantation rejection disease, including a compound represented by Formula 1 below or a pharmaceutically acceptable salt thereof and a microbiome strain as active ingredients.

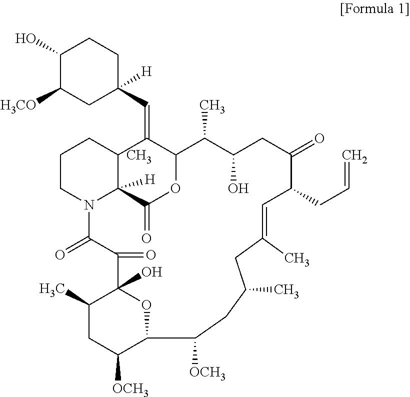

The present disclosure has also been made in an effort to provide a pharmaceutical composition for post-transplant immunosuppression, including the compound represented by Formula 1 above or a pharmaceutically acceptable salt thereof and a microbiome strain as active ingredients.

An exemplary embodiment of the present disclosure provides a pharmaceutical composition for prevention or treatment of transplantation rejection or transplantation rejection disease, including a compound represented by Formula 1 below or a pharmaceutically acceptable salt thereof and a microbiome strain as active ingredients.

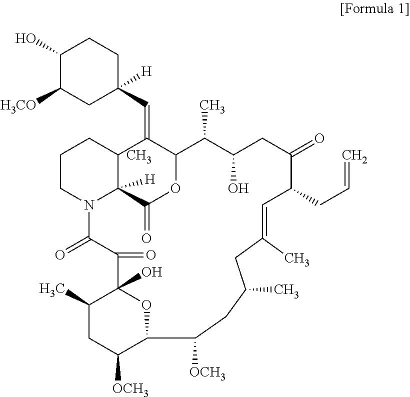

Another exemplary embodiment of the present disclosure provides a pharmaceutical composition for post-transplant immunosuppression, including the compound represented by Formula 1 above or a pharmaceutically acceptable salt thereof and a microbiome strain as active ingredients.

According to the exemplary embodiments of the present disclosure, it was confirmed that the combined administration of FK506 and a microbiome suppresses alloresponse and regulates the activity of T cells and B cells in an allogeneic transplantation cell model. Further, it was confirmed that in an acute graft-versus-host disease animal model, the combined administration reduces disease activity, increases probability of survival, and protects tissue damage. In addition, it was confirmed that the combined administration improves transplantation rejection diseases by reducing the activity of Th1 and Th17 and increasing the activity of Tregs, and thus can be usefully used as an immunomodulator for transplantation rejection. In addition, it was confirmed that the combination of FK506 and the microbiome inhibited the proliferation of alloresponse T cells, suppressed the expression of IL-17, and increased the expression of IL-10, an immunoregulatory factor reduced by treatment with the immunosuppressant. In addition, it was confirmed that the combination of FK506 and the microbiome decreased IL-17, increased the expression of IL-10 and IFN-γ, and increased the immunoregulatory cells even in stimulated patient-derived PBMCs, and thus can be usefully used as an immunomodulator for transplantation rejection.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a diagram confirming an alloresponse inhibitory effect by treating FK506 and a microbiome strain of the present invention in an allogeneic cell model.

FIG. 2 is a diagram illustrating quantification analysis of Th1 and Th17 activities by flow cytometry by treating FK506 and the microbiome strain of the present invention under T cell activity conditions (A: confirmation of Th1 activity, B: confirmation of Th17 activity).

FIG. 3 is a diagram illustrating analysis of the expression of cytokines in a culture medium by ELISA by treating FK506 and the microbiome strain of the present invention under T cell activity conditions (A: confirmation of INF-γ expression, B: confirmation of IL-17 expression).

FIGS. 4A1, 4A2, 4A3, 4B1 and 4B2 show diagrams confirming the regulation of T cell and B cell activities by flow cytometry by administering FK506 and the microbiome strain of the present invention to mice with allogeneic transplantation rejection (FIGS. 4A1 to 4A3: confirmation of T cell regulation, FIGS. 4B1 and 4B2: confirmation of B cell regulation).

FIG. 5 schematically illustrates an experimental process for evaluating an effect of FK506 and the microbiome strain of the present invention in mice with acute graft-versus-host disease.

FIG. 6 is a diagram confirming an effect of inhibiting graft-versus-host disease activity by administering FK506 and a Lactobacillus paracasei strain of the present invention to mice with acute graft-versus-host disease (A: confirmation of disease activity, B: confirmation of weight change, C: confirmation of probability of survival).

FIG. 7 is a diagram confirming an effect of inhibiting graft-versus-host disease activity by administering FK506 and a Lactobacillus rhamnosus strain of the present invention to mice with acute graft-versus-host disease (A: confirmation of disease activity, B: confirmation of weight change, C: confirmation of probability of survival).

FIGS. 8A and 8B show diagrams confirming inhibition of liver and intestinal tissue damage by H&E staining by administering FK506 and a microbiome strain of the present invention to mice with acute graft-versus-host disease (FIG. 8A: confirmation of staining results, FIG. 8B: quantification of staining results).

FIG. 9 is a diagram illustrating quantitative analysis of a T cell activity regulatory effect by flow cytometry by administering FK506 and a microbiome strain of the present invention to mice with acute graft-versus-host disease (A: quantification of Th1 activity, B: quantification of Th17 activity, C: quantification of Treg activity, D: quantification of CD8-INFγ cell activity).

FIG. 10 is a diagram confirming the proliferation of alloresponse T cells using CCK8 according to a combined treatment of FK506 and a microbiome strain of the present invention.

FIG. 11 is a diagram analyzing the expression of inflammation-related factors by ELISA under alloresponse conditions according to a combined treatment of FK506 and a microbiome strain of the present invention (A: confirmation of IL-17, B: confirmation of IL-10).

FIG. 12 is a diagram confirming the expression of immune factors by ELISA according to a combined treatment of FK506 and a microbiome under T cell stimulation conditions in hPBMC (A: confirmation of IL-17, B: confirmation of IL-10).

FIG. 13 is a diagram confirming the expression of IFN-γ by ELISA according to a combined treatment of FK506 and a microbiome under T cell stimulation conditions in hPBMC.

FIGS. 14A and 14B show diagrams confirming the expression of Treg by flow cytometry according to a combined treatment of FK506 and a microbiome under T cell stimulation conditions in hPBMC (FIG. 14A: flow cytometry result, FIG. 14B: quantification of Treg expression).

FIGS. 15A and 15B show diagrams confirming the expression of Breg by flow cytometry according to a combined treatment of FK506 and a microbiome under LPS stimulation conditions in hPBMC (FIG. 15A: flow cytometry result, FIG. 15B: quantification of Breg expression).

FIGS. 16A and 16B show diagrams confirming the expression of B10 by flow cytometry according to a combined treatment of FK506 and a microbiome under LPS stimulation conditions in hPBMC (FIG. 16A: flow cytometry result, FIG. 16B: quantification of B10 expression).

FIGS. 17A and 17B show diagrams confirming the expression of CD19 and IL-10 positive cells by flow cytometry according to a combined treatment of FK506 and a microbiome under LPS stimulation conditions in hPBMC (FIG. 17A: flow cytometry result, FIG. 17B: quantification of expression of CD19 and IL-10 positive cells).

### DETAILED DESCRIPTION

Hereinafter, exemplary embodiments of the present disclosure will be described in detail with reference to the accompanying drawings. In the following description, detailed descriptions of techniques well-known to those skilled in the art may be omitted. Further, in describing the present disclosure, the detailed description of associated known functions or constitutions will be omitted if it is determined to unnecessarily make the gist of the present disclosure unclear. Terminologies used herein are terminologies used to properly express preferred exemplary embodiments of the present disclosure, which may vary according to a user, an operator's intention, or customs in the art to which the present disclosure pertains.

Accordingly, definitions of the terminologies need to be described based on contents throughout this specification. Throughout the specification, unless explicitly described to the contrary, when a certain part “comprises” a certain component, it will be understood to imply the inclusion of stated elements but not the exclusion of any other elements.

The present disclosure provides a pharmaceutical composition for prevention or treatment of transplantation rejection or transplantation rejection disease, including a compound represented by Formula 1 below or a pharmaceutically acceptable salt thereof and a microbiome strain as active ingredients.

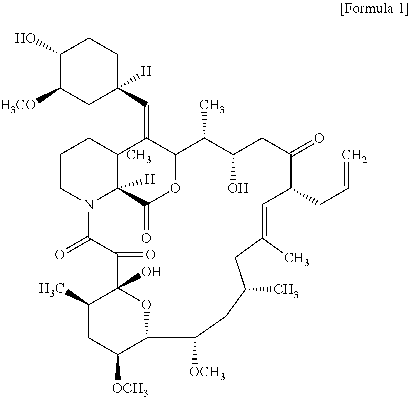

As used herein, the term “prevention” refers to any action that suppresses the symptoms of a specific disease or delays its progression by administering the composition of the present disclosure.

As used herein, the term “treatment” refers to any action that improves or beneficially changes the symptoms of a specific disease by administering the composition of the present disclosure.

The compound of Formula 1 of the present disclosure is also called FK506 or Tacrolimus, and is an immunosuppressant produced by Streptomyces taukuaensis.

In addition, the compound represented by Formula 1 according to the present disclosure may be used in the form of a salt, preferably a pharmaceutically acceptable salt.

The term “pharmaceutically acceptable salt” means these salts which are intended for use in contact with human and lower animal tissues without excessive toxicity, irritation, allergic reaction, and things similar thereto, within the scope of sound medical judgment, and are proportional to a reasonable advantage/disadvantage ratio. For example, the pharmaceutically acceptable salts are described in detail in S. M. Berge et al., J. Pharmaceutical Sciences, 1977, 66, 1-19, which is incorporated herein by reference. The pharmaceutically acceptable salts of the compound of the present disclosure include salts derived from suitable inorganic and organic acids and bases. Examples of pharmaceutically acceptable and non-toxic acid addition salts include amino group salts that are formed by inorganic acids such as hydrochloric acid, hydrobromic acid, phosphoric acid, sulfuric acid and perchloric acid, or organic acids such as acetic acid, oxalic acid, maleic acid, tartaric acid, citric acid, succinic acid, or malonic acid, or formed using other methods used in the art, such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecyl sulfate, ethane sulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate, and similar salts thereto.

Salts derived from suitable bases include alkali metal, alkaline earth metal, ammonium, and N⁺(C₁₋₄alkyl)₄ salts. Representative alkali or alkaline earth metal salts include sodium, lithium, potassium, calcium, magnesium, and similar salts thereto. In addition, the pharmaceutically acceptable salts include non-toxic ammonium, quaternary ammonium, and amine cations formed using counter ions such as halide, hydroxide, carboxylate, sulfate, phosphate, nitrate, lower alkyl sulfonate, and aryl sulfonates, at the right time.

The pharmaceutical composition of the present disclosure may further include an adjuvant in addition to the active ingredients. The adjuvant may be used with any adjuvant known in the art without limitation, but further include, for example, a Freund's complete adjuvant or an incomplete adjuvant to increase the effect thereof.

The pharmaceutical composition according to the present disclosure may be prepared in the form of incorporating the active ingredients into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, aerosols, etc., external preparations, suppositories, or sterile injectable solutions according to each conventional method, and preferably injectable solutions (injections), but is not limited thereto.

The pharmaceutical composition of the present disclosure may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one excipient, for example, starch, calcium carbonate, sucrose, lactose, gelatin, etc. with the active ingredients. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used simple diluents, such as water and liquid paraffin. Formulations for parenteral administration include sterile aqueous solutions, non-aqueous solvents, suspensions, emulsions, lyophilized agents, and suppositories. As the non-aqueous solvent and the suspension, propylene glycol, polyethylene glycol, vegetable oil such as olive oil, injectable ester such as ethyl oleate, etc. may be used. As the base of the suppository, witepsol, Tween 61, cacao butter, laurinum, glycerogelatin, etc. may be used.

The pharmaceutical composition of the present disclosure may be administered to a subject through various routes. All methods of administration may be expected, and the pharmaceutical composition may be administered, for example, oral, intravenous, intramuscular, subcutaneous, intraperitoneal, and intraarticular injection.

The dose of the pharmaceutical composition according to the present disclosure is selected in consideration of the age, body weight, sex, and physical conditions of the subject. It is obvious that the concentration of the active ingredients included in the pharmaceutical composition may be variously selected according to a subject, and preferably included in the pharmaceutical composition at a concentration of 0.01 to 5,000 μg/ml. When the concentration is less than 0.01 μg/ml, pharmaceutical activity may not be shown, and when the concentration exceeds 5,000 μg/ml, toxicity to the human body may be exhibited.

According to an exemplary embodiment of the present disclosure, the transplantation rejection may be transplantation rejection selected from the group consisting of cells, blood, tissues, and organs, and the transplantation rejection may be selected from the group consisting of rejection for bone marrow transplantation, heart transplantation, corneal transplantation, intestinal transplantation, liver transplantation, lung transplantation, pancreas transplantation, kidney transplantation, and skin transplantation, but is not limited thereto.

According to an exemplary embodiment of the present disclosure, the transplantation rejection may include graft-versus-host disease (GVHD).

According to an exemplary embodiment of the present disclosure, the microbiome strain may be selected from the group consisting of Lactobacillus rhamnosus, Lactobacillus paracasei, Lactobacillus gasseri and Lactobacillus acidophilus.

As used herein, the term ‘microbiome’ refers to the entire genetic information of microorganisms inhabiting the human body or the microorganisms themselves, and is a compound word of microbiota, which are microorganisms that inhabit and coexist in the human body, and genome. It is known that the number of human microbiomes is at least twice greater than the number of pure human cells and the number of genes is at least 100 times greater therethan, and the microbiome refers to a field that can be widely used in research on the microbial environment within the human body, such as development of new drugs and treatment of incurable diseases by analyzing the principles of formation of beneficial and harmful bacteria and the relation between diseases.

According to an exemplary embodiment of the present disclosure, the composition may increase the probability of survival of a graft.

According to an exemplary embodiment of the present disclosure, the composition may regulate immune cells, and the immune cells may be cells selected from the group consisting of Th1, Th17, Treg, IL-10 producing B cell, plasma B cell, alloresponse T cell, Breg, B10 and CD19+IL-10+cell.

According to an exemplary embodiment of the present disclosure, the regulating of the immune cells may be inhibiting the activity of Th1, Th17, plasma B cell or alloresponse T cell, increasing the expression of immunoregulatory cells decreased by treatment of the immunosuppressant, and increasing the activity of cells selected from the group consisting of Treg, IL-10 producing B cell, Breg, B10 and CD19+IL-10+cell.

According to an exemplary embodiment of the present disclosure, the composition may protect tissue damage, and the tissue may be a tissue selected from the group consisting of bone marrow, heart, cornea, intestine, liver, lung, pancreas, kidney, and skin.

According to an exemplary embodiment of the present disclosure, the composition may decrease the expression of IL-17.

According to an exemplary embodiment of the present disclosure, the composition may increase the expression of IL-10 or IFN-γ.

In addition, the present disclosure provides a pharmaceutical composition for post-transplant immunosuppression, including the compound represented by Formula 1 above or a pharmaceutically acceptable salt thereof and a microbiome strain as active ingredients.

Hereinafter, the present disclosure will be described in more detail through Examples. These Examples are to explain the present disclosure in more detail, and it will be apparent to those skilled in the art that the scope of the present disclosure is not limited to these Examples.

### <Example 1> Fabrication of Allotransplantation Rejection Model and Confirmation of Alloresponse Inhibition (In Vitro)

To confirm an effect of the combination of FK506 and a microbiome of the present invention on transplantation rejection, an allogeneic cell model was used. Specifically, in a 96 well plate, 2×10⁵ CD4+T cells from a normal responder (Bclb/c) per well were mixed and co-cultured with 2×10⁵ splenocytes from which T cells derived from an irradiated responder (syngenic) or donor (C57BL/6, stimulator, allogeneic) were removed. During co-culture, an allogeneic model was treated with five types of Lactobacillus bacteria, Lactobacillus acidophilus (LA), L. reuteri (LRe), L. rhamnosus (LRh), L. paracasei (LPC) or L. casei (LC) alone at 1, 10, and 100 μg/ml, or administered in combination with 0.3 mg/ml of FK506. As a control group, a syngenic transplantation group (Syn group) and an allogeneic transplantation group (Allo group) were used. Thereafter, in order to confirm alloresponses in mice of each group, T cell proliferation responses (alloresponses) were confirmed in the cultured cells using a 3H thymidine incorporation method.

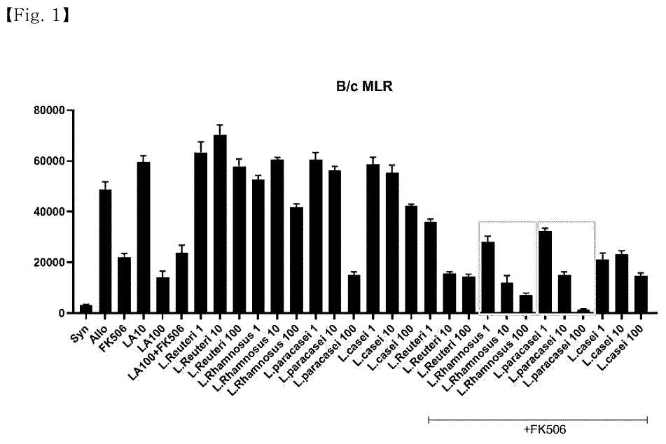

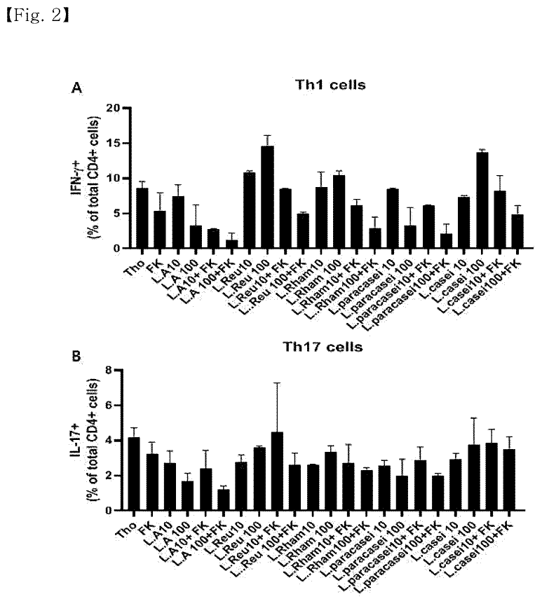

As a result, as illustrated in FIG. 1, T cell proliferation increased in an Allo group (positive control), and in a group administered with FK506 alone, T cell proliferation was suppressed. In addition, Lactobacillus rhamnosus or Lactobacillus paracasei was identified as the microbiome showing a synergistic effect on T cell proliferation inhibition of FK506. In a group where 100 μg/ml of each microbiome was administered in combination with FK506, T cell proliferation was significantly suppressed compared to administration of FK506 alone, and thus it was confirmed that the combination of FK506 and Lactobacillus rhamnosus or Lactobacillus paracasei effectively inhibited alloresponse.

### <Example 2> Confirmation of T Cell Activity Regulation by Combination of Microbiome Strain and FK506

It was confirmed whether the combination of FK506 and the microbiome of the present invention regulated the activity of T cells. Specifically, mouse-derived spleen tissue was extracted, T cells in the spleen tissue were stimulated with CD3, and the T cells were activated. During CD3 stimulation, in the same manner as in Example 1, five types of Lactobacillus strains and FK506 were treated alone or in combination and cultured for 3 days. After completion of culture, the activities of autoimmune pathogenic T cells, Th1 and Th17, were analyzed by flow cytometry. In addition, a culture medium was obtained at the end of the culture, and the secretion of INF-γ and IL-17, which were inflammatory cytokines secreted by autoimmune pathogenic T cells in the culture medium, was measured by ELISA.

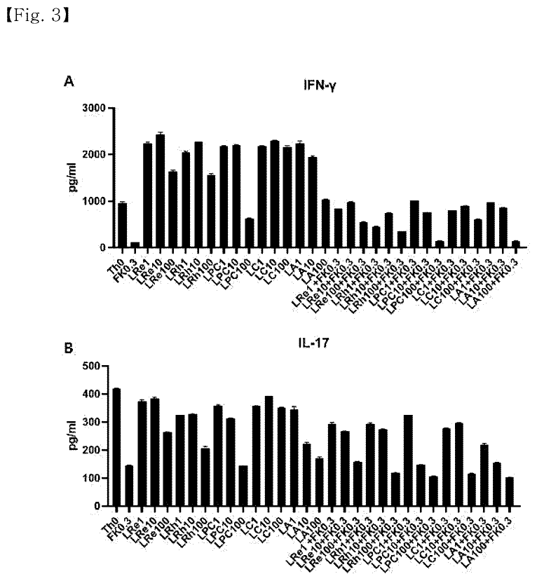

As a result, as illustrated in FIG. 2, it was confirmed that compared to the control group, in the group treated with Lactobacillus acidophilus, Lactobacillus rhamnosus or Lactobacillus paracasei in combination with FK506, the activities of Th1 and Th17 were significantly suppressed. In addition, it was confirmed that in the group treated with Lactobacillus acidophilus or Lactobacillus paracasei in combination with FK506, the secretion of IFN-γ and IL-17 in the culture medium was significantly reduced (FIG. 3).

### <Example 3> Confirmation of Simultaneous Regulation of T and B Cells by Combination of Microbiome Strain and FK506

It was confirmed whether the combination of FK506 and the microbiome of the present invention regulated simultaneously T cells and B cells. Specifically, splenocytes were isolated from a transplantation disease animal model (acute graft-versus-host disease (aGVHD)), and CD3 (0.5 ug/ml) and LPS (100 ng/ml) were treated as an activation condition for T cells and B cells to activate the T cells and B cells. Thereafter, in an alloresponse, Lactobacillus rhamnosus or Lactobacillus paracasei, in which the T cell regulatory effect was confirmed, was treated in combination with FK506 0.3 nM at a concentration of 100 μg/ml and cultured for 24 hours, and then the activities of immune cells Th1, Th17, Treg, IL-10 producing B cells, and plasma B cells (plasma cells) were analyzed by flow cytometry.

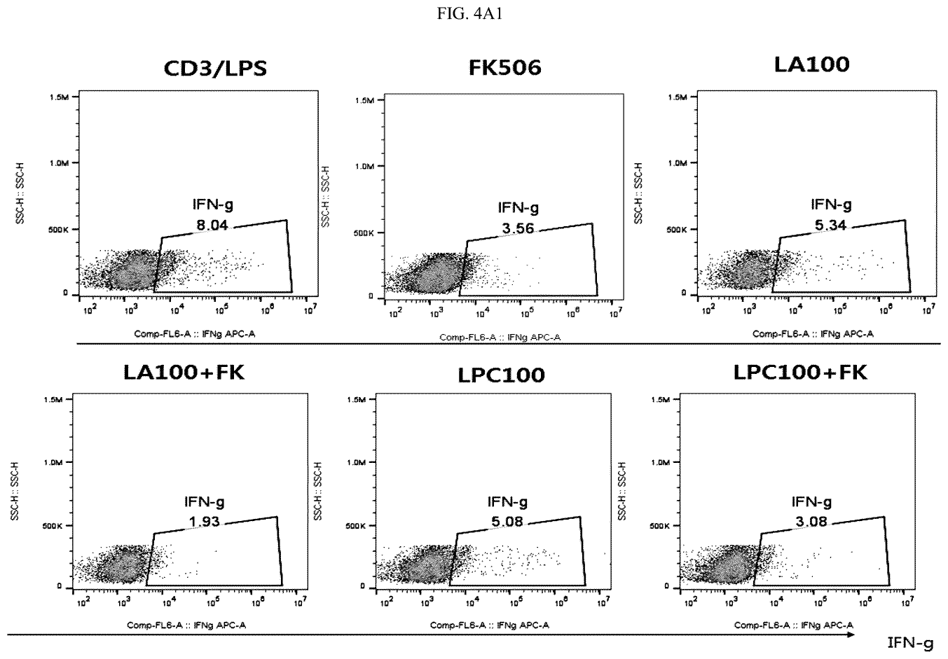

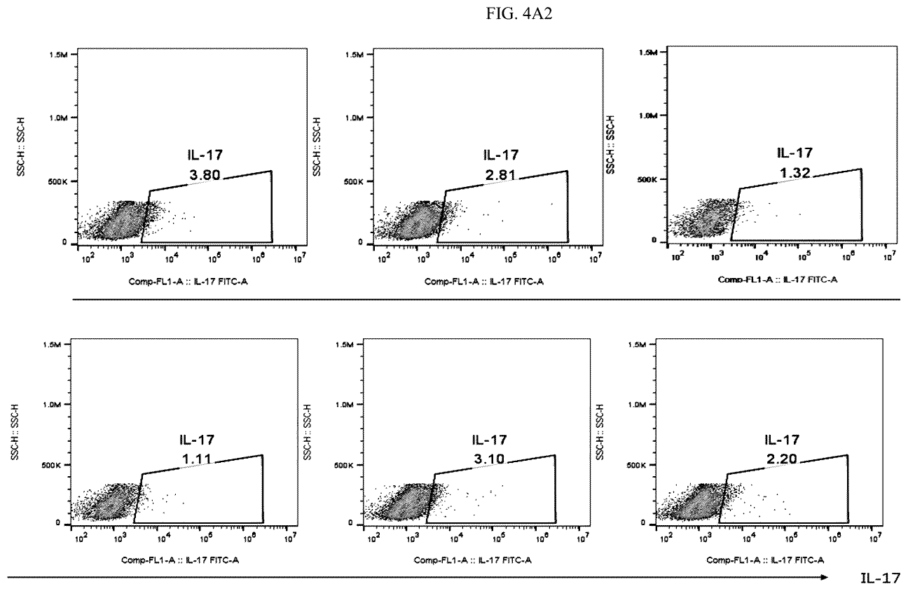

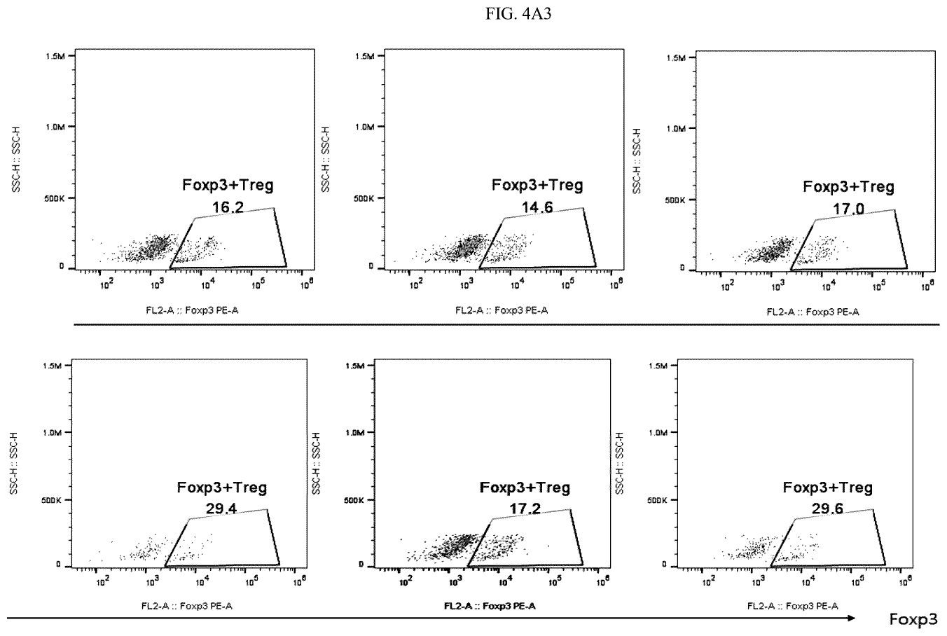

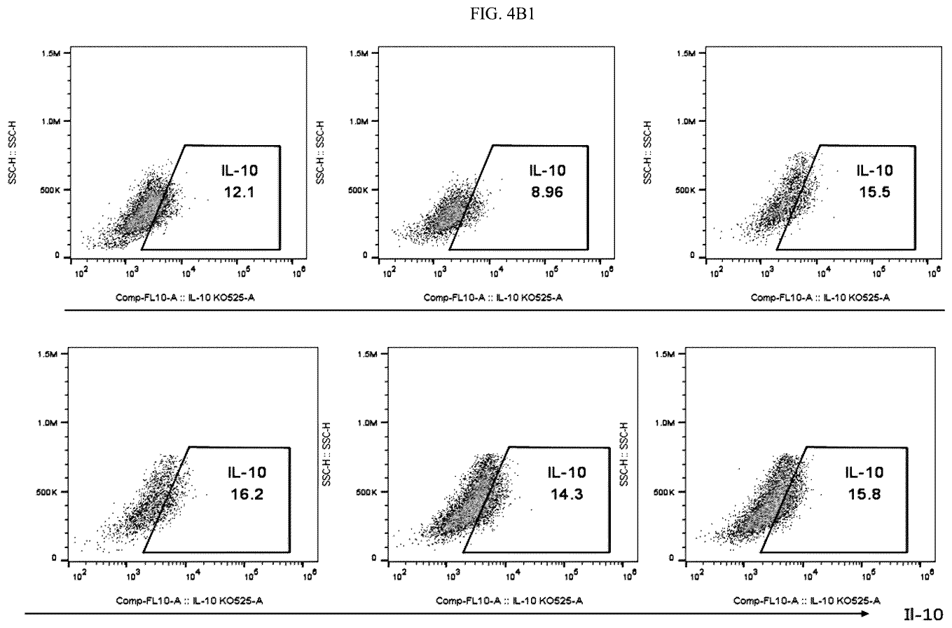

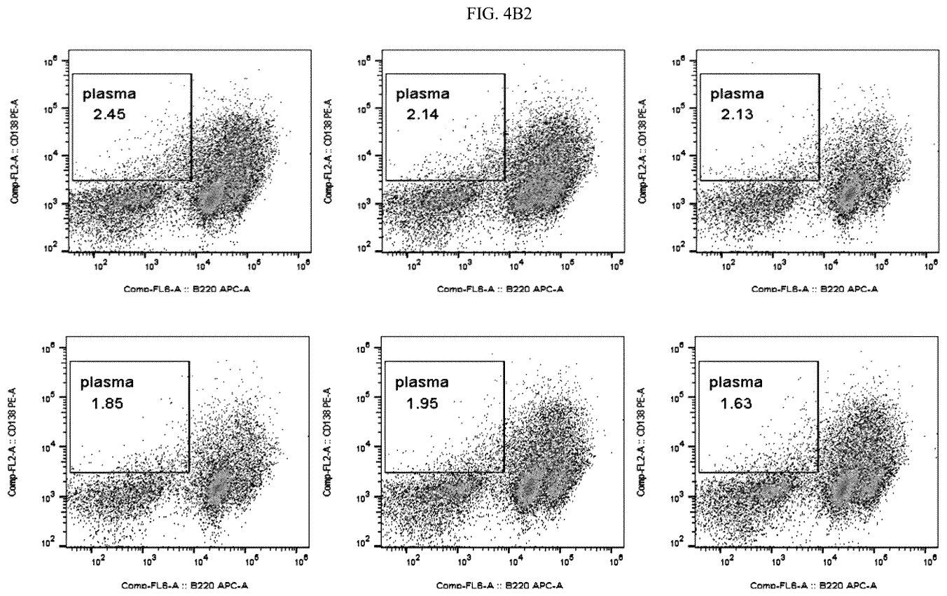

As a result, as illustrated in FIGS. 4A1, 4A2 and 4A3, compared to the control group, in a group treated with FK506 and Lactobacillus acidophilus (LA) or Lactobacillus paracasei (LPC), the activity of autoimmune pathogenic T cells, Th1 and Th17, was significantly suppressed, and the activity of immune regulatory cells, Tregs, was significantly increased, and thus a synergistic effect of FK506 and the microbiome in regulating immune cells was confirmed. In addition, the activity of IL-10 producing B cells was significantly increased, and the activity of pathogenic plasma B cells was significantly decreased (FIGS. 4B1 and 4B2), and thus the co-regulation effect of T cells and B cells was confirmed.

### <Example 4> Confirmation of Disease Activity Control in Acute Graft-Versus-Host Disease Animal Model

It was confirmed whether the combination of FK506 and Lactobacillus rhamnosus or Lactobacillus paracasei of the present invention improved the disease activity of acute graft-versus-host disease (aGVHD). Specifically, in order to fabricate an aGVHD model, a responder mouse Balb/c (H-2k/d) was subjected to total body irradiation (TBI) of 690 cGy, bone marrow stem cells and splenocytes were isolated from the femur and tibia of a donor mouse C57BL/6 (H-2k/b), and then 5×10⁶ bone marrow (BM) stem cells and 5×10⁶ splenocytes were transplanted into the responder mouse Balb/c (H-2k/d). Thereafter, after the occurrence of aGVHD, 5 mg/kg of FK506 and 400 mg/kg each of Lactobacillus rhamnosus (LR) or Lactobacillus paracasei (LP) were administered orally once daily for 4 weeks. Disease activity, body weight, and probability of survival of mice in each group were measured once a week to confirm whether the disease activity was suppressed (FIG. 5). As control groups, a syngenic (syn) group transplanted with syngenic cells; a vehicle group, in which aGVHD was induced and saline was injected; and groups administered with FK506, Lactobacillus rhamnosus, and Lactobacillus paracasei alone were used.

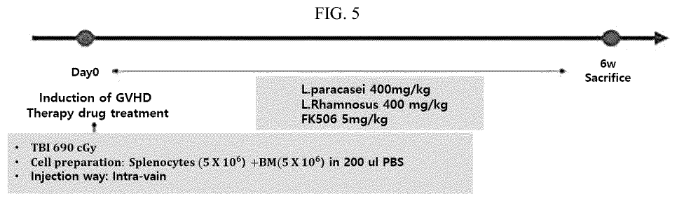

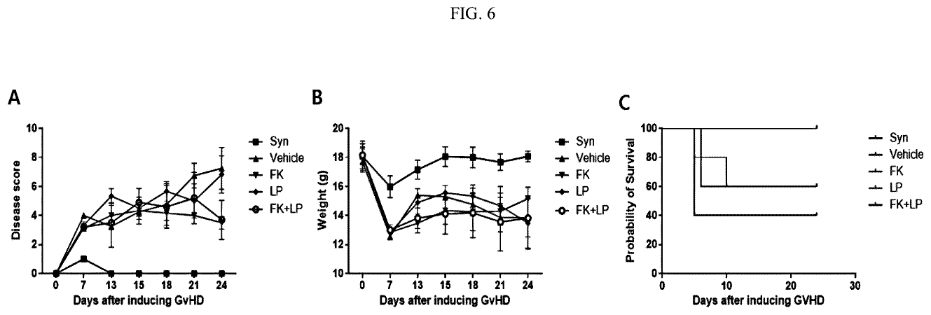

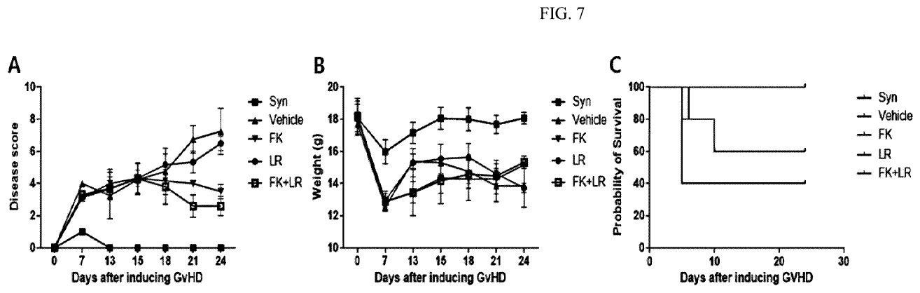

As a result, as illustrated in FIGS. 6 and 7, it was confirmed that compared to the syngenic group, in the vehicle group, the disease activity significantly increased, the body weight decreased due to aGVHD induction, and the probability of survival of mice decreased to 40% 5 days after aGVHD induction. However, it was confirmed that in the group co-administered with FK506 and Lactobacillus rhamnosus or Lactobacillus paracasei, compared to the vehicle group, the disease activity significantly decreased, and the body weight increased. In addition, it was confirmed that the probability of survival of mice was maintained at 100% until the end of the experiment, and thus it was confirmed that the disease activity was suppressed. In addition, it was confirmed that in the groups administered with FK506, Lactobacillus rhamnosus, and Lactobacillus paracasei alone, compared to the vehicle group, the disease activity decreased, and the body weight increased, but the probability of survival of each group at the end of the experiment was 60%. Thus, it was confirmed that the combined administration of FK506 and Lactobacillus rhamnosus; or Lactobacillus paracasei had an excellent effect of inhibiting the disease activity of aGVHD.

### <Example 5> Confirmation of Tissue Damage Protection in Acute Graft-Versus-Host Disease Animal Model

It was confirmed whether the combination of FK506 and Lactobacillus rhamnosus; or Lactobacillus paracasei of the present invention protected the tissue damage in an aGVHD animal model. Specifically, mice of each group in Example 4 were humanely sacrificed at the end of the experiment, liver, and intestine tissues, which were target organs damaged in GVHD, were obtained, and treated with hematoxylin and eosin (H&E) staining to confirm tissue damage, which was expressed as a histological score.

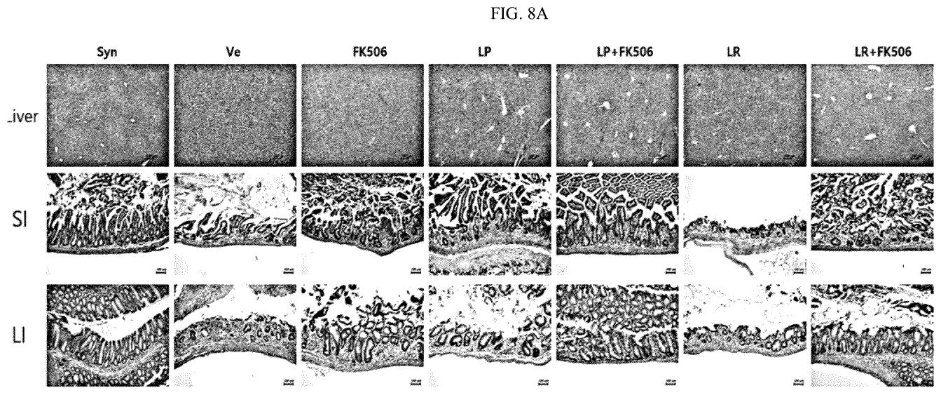

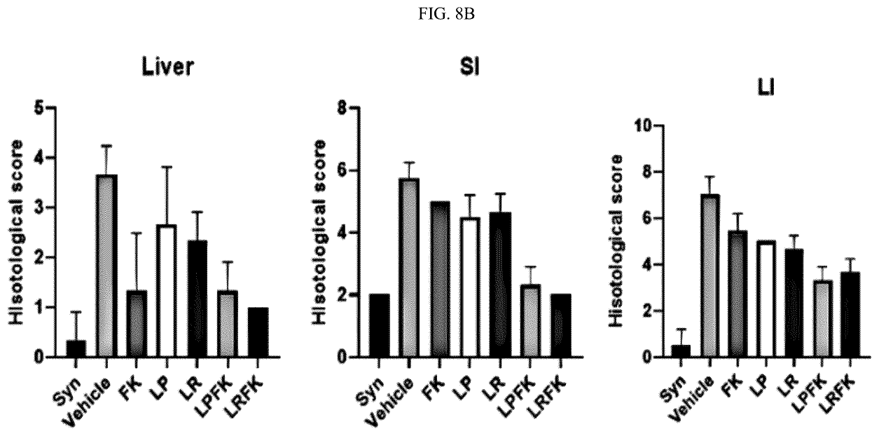

As a result, as illustrated in FIGS. 8A and 8B, it was confirmed that compared to the syngenic group, in the vehicle group, damage to the liver and tissues increased, and the histological index increased, but in the group co-administered with FK506 and Lactobacillus rhamnosus; or Lactobacillus paracasei, the histological index was significantly reduced, thereby protecting tissue damage.

### <Example 6> Confirmation of Immune Cell Regulation in Acute Graft-Versus-Host Disease Animal Model

It was confirmed whether the combination of FK506 and Lactobacillus rhamnosus; or Lactobacillus paracasei of the present invention regulated immune cells in an aGVHD animal model. Specifically, spleen tissue was obtained from mice of each group sacrificed in Example 5, and the activities of immune cells Th1, Th17, Tregs, and CD8-INFγ cells were analyzed by flow cytometry.

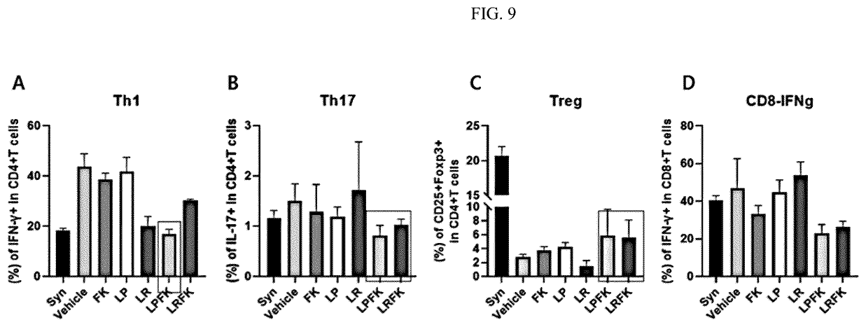

As a result, as illustrated in FIG. 9, it was confirmed that compared to the syngenic group, in the vehicle group, the activities of Th1 and Th17 increased, and the activity of Tregs decreased, but in the group co-administered with FK506 and Lactobacillus rhamnosus; or Lactobacillus paracasei, the increased activities of Th1 and Th17 were significantly reduced, and the activity of Tregs was significantly increased.

### <Example 7> Confirmation of Regulation of Allo Response Cells and Inflammatory Factors

### <7-1> Confirmation of Inhibition of Allo Response T Cell Proliferation

It was confirmed whether a combination of FK506 and a microbiome of the present invention inhibited the proliferation of allo response T cells. Specifically, the proliferation of allo response T cells was confirmed for FK506 alone (1 nM) and a combination of FK506 (1 nM) and L. gasseri (10 μg/ml); L. gasseri (10 μg/ml) and L. acidophilus (10 μg/ml); two microbiomes (each 10 μg/ml) and zinc (50 μM); or two microbiomes (each 10 μg/ml), zinc (50 μM), vitamin B9, and vitamin B12 (B9+B12 20 μM). Specifically, different human peripheral blood mononuclear cells (hPBMCs) were inoculated at a ratio of 1:1 (each concentration of 1×10⁵ cells) on a 96 well plate coated with a concentration of anti CD3 0.5 μg/ml, treated with the combination in hPBMCs in which allo conditions were induced, and then cultured for 72 hours. Thereafter, 10 μl of CCK8 was treated to each well and reacted for 4 hours, and then the cell proliferation response was measured by absorbance at 450 nm. As a control group, a Vehicle group in which the active ingredient was replaced with a solvent was used.

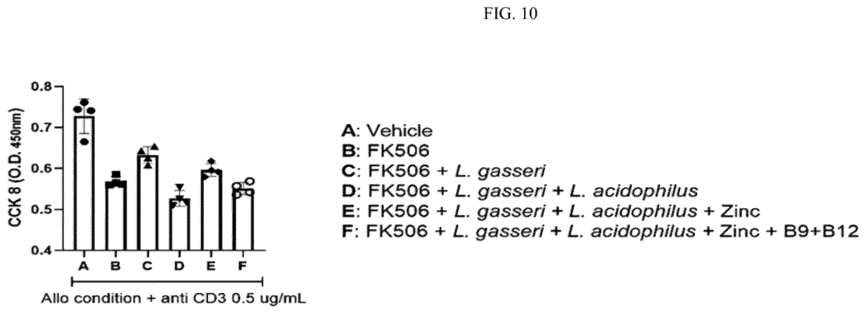

As a result, as illustrated in FIG. 10, it was confirmed that compared with the Vehicle group, the proliferation of allo response T cells was inhibited in all treated groups, and particularly, in a group co-administered with FK506 and L. gasseri and L. acidophilus; and a group co-administered with microbiomes, zinc, vitamin B9, and vitamin B12, an allo response T cell inhibitory effect was excellent.

### <7-2> Confirmation of Regulation of Inflammation-Related Factors Under Allo Response Conditions

It was confirmed whether the combination of FK506 and the microbiome of the present invention regulated the expression of inflammation-related factors under allo response conditions. Specifically, a culture medium of each cell cultured in Example 7-1 was obtained, and the amounts of IL-17 as an inflammatory factor and IL-10 as an inflammation regulator in the culture medium were analyzed by ELISA.

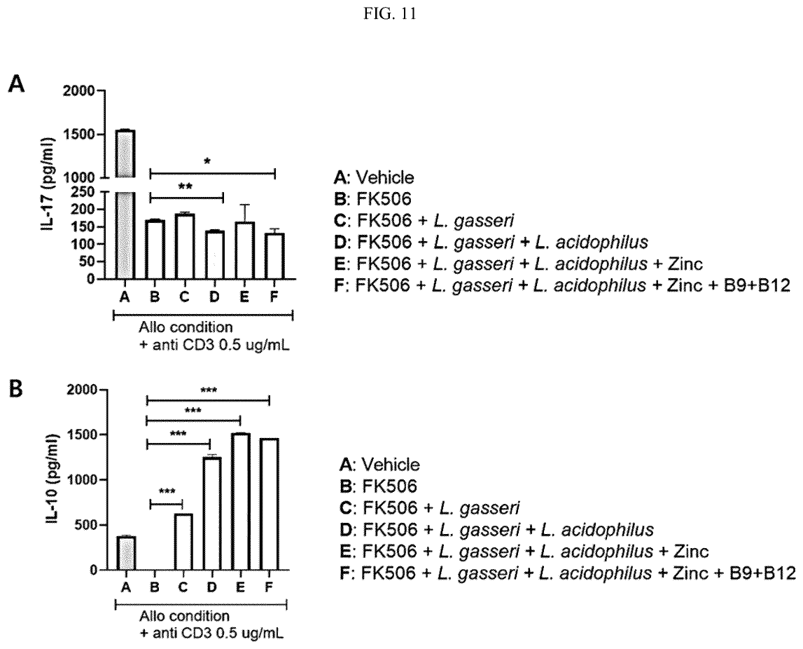

As a result, as shown in FIG. 11, it was confirmed that the amount of IL-17 was significantly decreased in all treated groups, compared to the Vehicle group. In addition, compared to the Vehicle group, IL-10 was significantly decreased in the group treated with FK506 alone, and treatment with the immunosuppressant decreased IL-10, but in all groups treated with the combination of FK506 and the microbiomes, the expression of IL-10 was significantly increased.

### <Example 8> Confirmation of Immunoregulatory Effect in hPBMC According to T Cell Stimulation

### <8-1> Confirmation of IL-17, IL-10, and IFN-γ Regulation Effects Under T Cell Stimulation Conditions

It was confirmed whether the combination of FK506 and the microbiomes of the present inention regulated IL-17 and IL-10 in human peripheral blood mononuclear cells (hPBMCs) under T cell stimulation conditions. Specifically, PBMCs isolated from human peripheral blood were treated with anti-CD3 at a concentration of 2 μg/ml to activate T cells. Thereafter, the PBMCs were treated with FK506 alone (1 nM) and a combination of FK506 (1 nM) and L. gasseri (10 μg/ml); L. gasseri (10 μg/ml) and L. acidophilus (10 μg/ml); two microbiomes (each 10 μg/ml) and zinc (50 μM); or two microbiomes (each 10 μg/ml), zinc (50 μM), vitamin B9, and vitamin B12 (B9+B12 20 μM) and cultured for 24 hours. After completion of the culture, the cell culture medium was obtained, and the amounts of IL-17 and IL-10 in the culture medium were measured by ELISA.

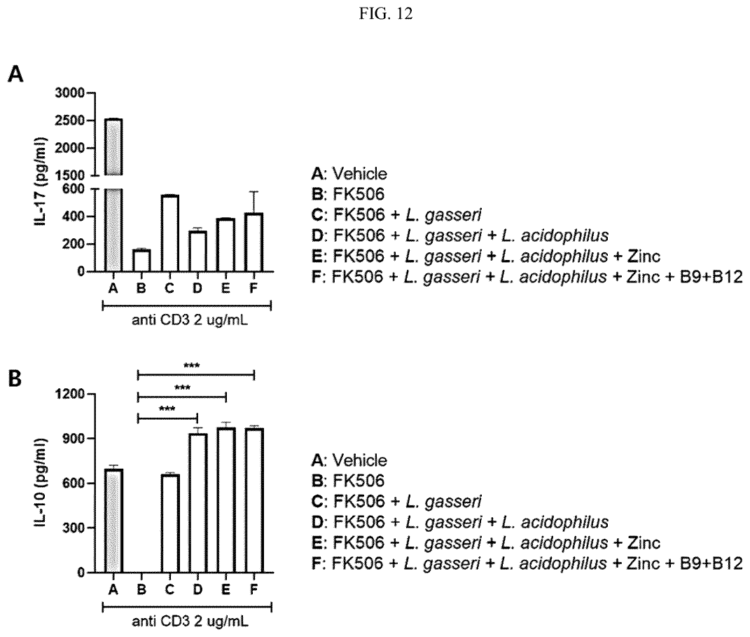

As a result, as shown in FIG. 12, it was confirmed that the expression of IL-17 was significantly decreased in all treated groups, compared to the Vehicle group. In addition, in the FK506-alone treated group, IL-10 was not expressed, but in all the groups co-administered with FK506 and the microbiomes, the amount of FK506 was increased, and particularly, in the group co-administered with FK506 and L. gasseri and L. acidophilus; microbiomes and zinc; or microbiomes, zinc, vitamin B9, and vitamin B12, the amount of FK506 was significantly increased.

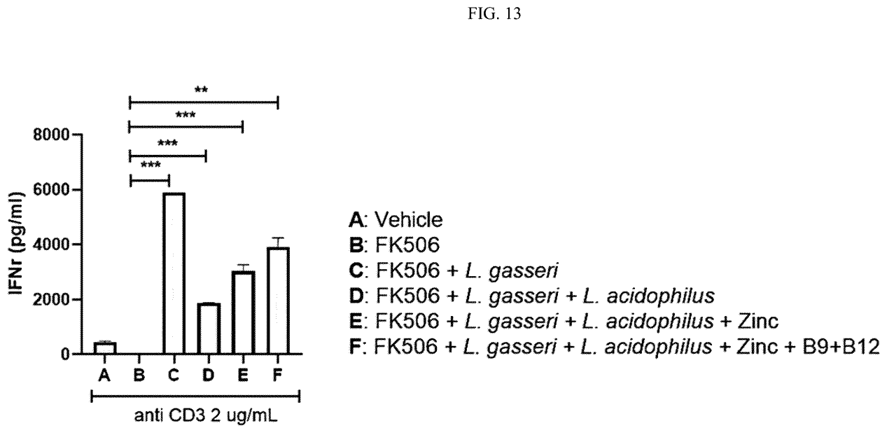

In addition, as a result of confirming the amount of IFN-γ, it was confirmed that compared with the Vehicle group, in the FK506-alone treated group, the amount of IFN-γ was significantly decreased, but in all the groups co-administered with FK506 and the microbiomes, the amount of IFN-γ was significantly increased, and particularly, in the group co-administered with FK506 and L. gasseri, the amount of IFN-γ was significantly increased compared to other treated groups (FIG. 13).

### <8-2> Confirmation of Treg Regulatory Effect Under T Cell Stimulation Conditions

It was confirmed whether the combination of FK506 and the microbiomes of the present invention regulated the expression of Treg reduced by treatment with the immunosuppressant. Specifically, cells in each group of Example 8-1 were obtained, and expression of Treg was confirmed by flow cytometry.

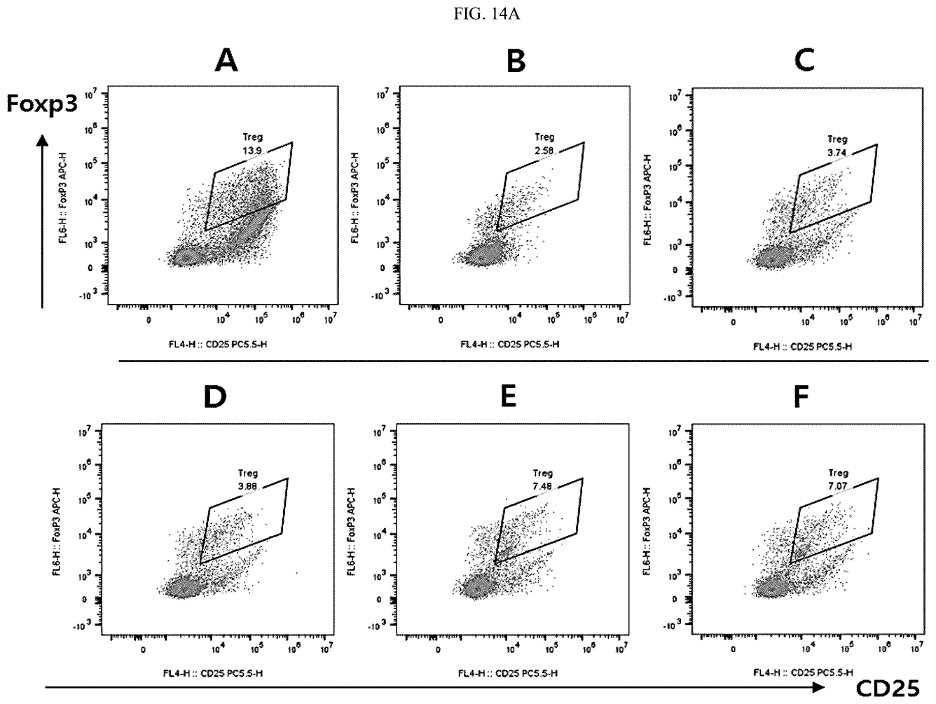

As a result, as shown in FIGS. 14A and 14B, it was confirmed that compared to the Vehicle group, in the group treated with the immunosuppressant FK506 alone, the expression of Treg was significantly decreased, but in all the groups co-administered with FK506 and the microbiomes, the expression of Treg was significantly increased, and particularly, in the group co-administered with two microbiomes and zinc; or two microbiomes, zinc, vitamin B9, and vitamin B12, the expression of Treg was significantly increased.

### <Example 9> Confirmation of Immune Cell Regulatory Effect in hPBMC According to LPS Stimulation

The immune cell regulatory effect of the combination of FK506 and the microbiomes of the present invention in hPBMC according to LPS was confirmed. Specifically, PBMCs isolated from human peripheral mononuclear cells were treated with LPS at a concentration of 100 ng/ml to activate immune cells. Thereafter, FK506 alone; or a combination of FK506 and L. gasseri; L. gasseri and L. acidophilus; microbiomes and zinc; or microbiomes, zinc, vitamin B9, and vitamin B12 was treated and cultured for 24 hours. After completion of culture, cells in each group were obtained, and the expression of immune B cells Breg and B10 cells, and CD19-positive and IL-10-positive cells was confirmed using flow cytometry.

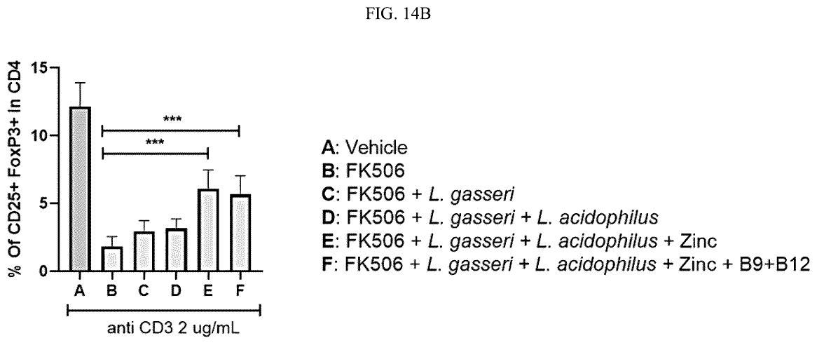

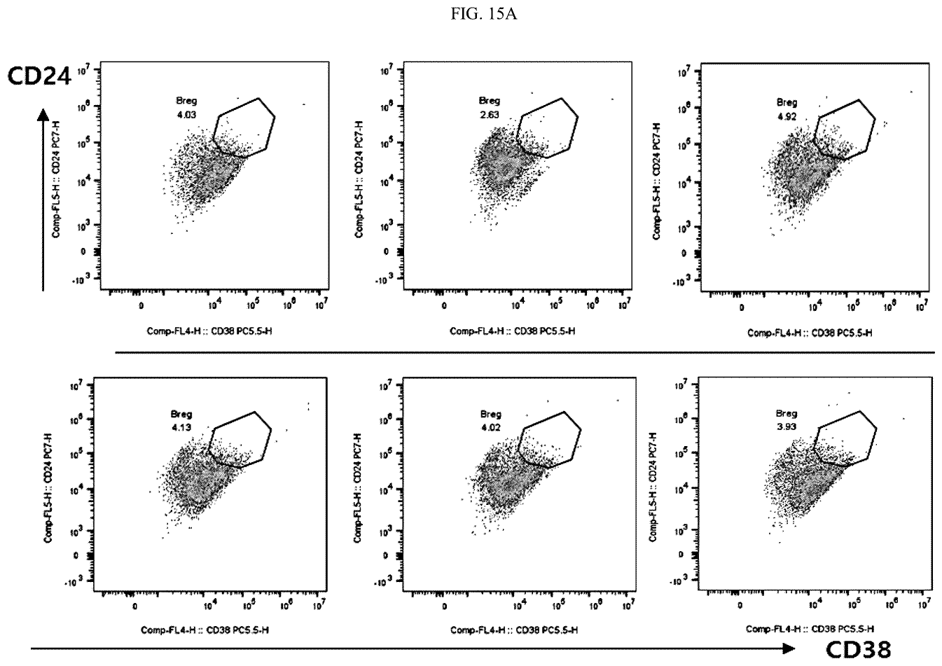

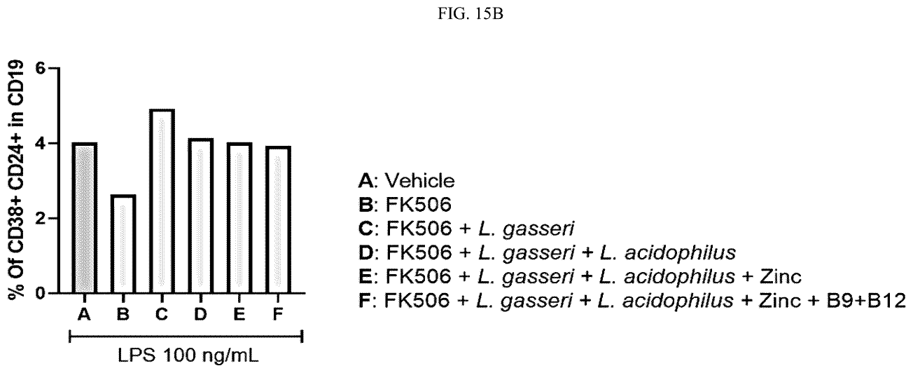

As a result, as shown in FIGS. 15A and 15B, compared to the Vehicle group, in the group treated with FK506 alone, the expression of Breg was significantly decreased, but in all the groups co-treated with microbiomes, the expression of Breg was increased.

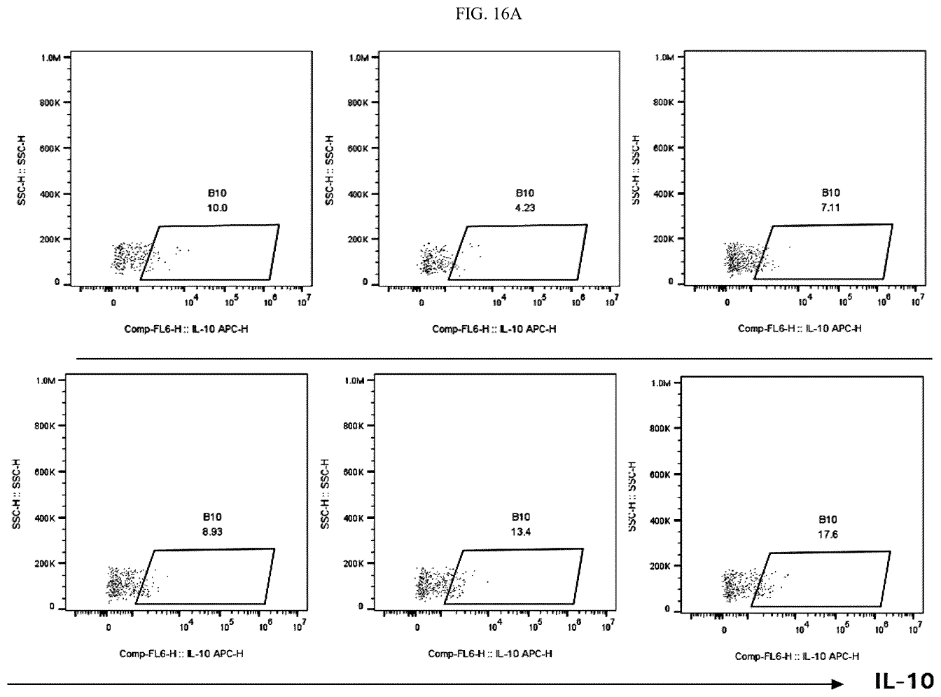

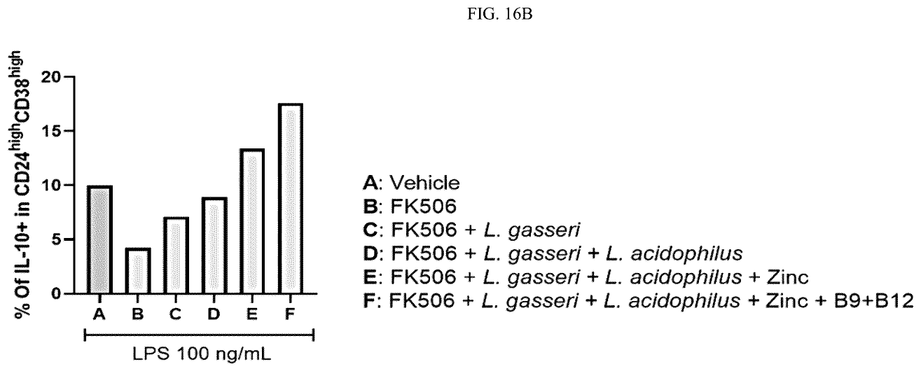

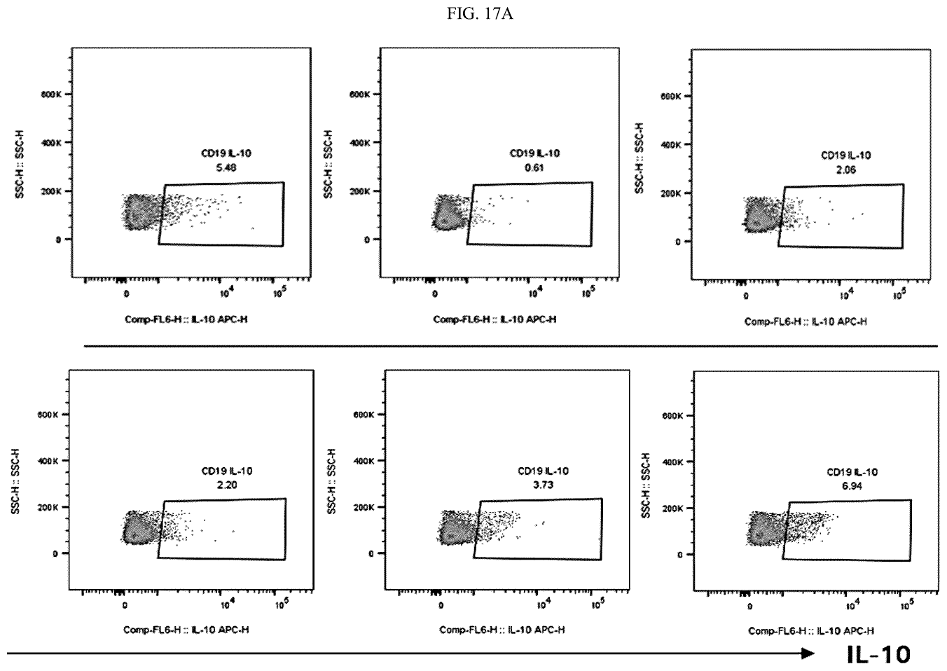

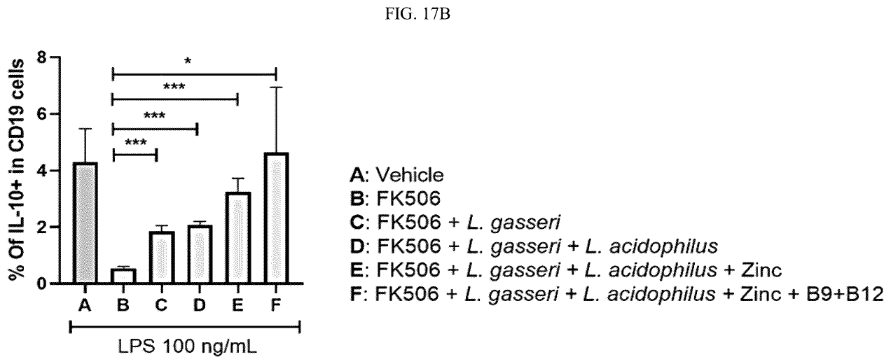

In addition, it was confirmed that in the group treated with FK506 alone, the expression of B10 was decreased, and in all the groups co-treated with the microbiomes and FK506, the expression of decreased B10 was recovered, and particularly, in the group co-treated with two microbiomes, zinc, vitamin B9, and vitamin B12, the B10 cells were significantly increased (FIGS. 16A and 16B). In addition, even in the CD19-positive and IL-10-positive cells, the results similar to the B10 cells were shown, and the CD19-positive and IL-10-positive cells were significantly increased in the group co-treated with FK506 and two microbiomes, zinc, vitamin B9, and vitamin B12 (FIGS. 17A and 17B).

Accordingly, it was confirmed that the combined administration of FK506 and the microbiome of the present disclosure suppressed the alloresponse and regulated the activities of T cells and B cells in the allogeneic transplantation cell model. Further, it was confirmed that in the acute graft-versus-host disease animal model, the combined administration reduced the disease activity, increased the probability of survival, and protected the tissue damage. Further, it was confirmed that transplantation rejection disease may be improved by reducing the activities of Th1 and Th17 and increasing the activity of Tregs. In addition, it was confirmed that the combination of FK506 and the microbiomes inhibited the proliferation of alloresponse T cells, suppressed the expression of IL-17, and increased the expression of IL-10, an immunoregulatory factor reduced by treatment with the immunosuppressant. In addition, it was confirmed that the combination of FK506 and the microbiomes decreased IL-17, increased the expression of IL-10 and IFN-γ, and increased the immunoregulatory cells even in stimulated patient-derived PBMCs.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method for treating transplantation rejection or transplantation rejection disease, comprising administering to a subject in need thereof a pharmaceutical composition comprising a compound represented by Formula 1 below or a pharmaceutically acceptable salt thereof and a microbiome strain as active ingredients

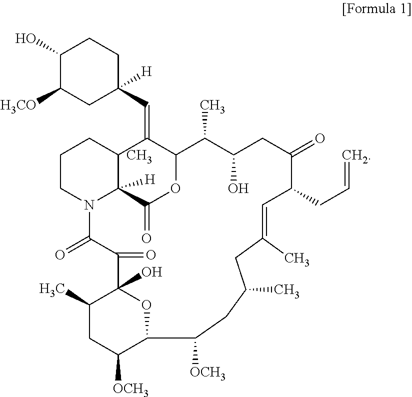

2. The method of claim 1, wherein the transplantation rejection is transplantation rejection selected from the group consisting of cells, blood, tissues and organs.

3. The method of claim 2, wherein the transplantation rejection is selected from the group consisting of rejection of bone marrow transplantation, heart transplantation, corneal transplantation, intestinal transplantation, liver transplantation, lung transplantation, pancreas transplantation, kidney transplantation, and skin transplantation.

4. The method of claim 1, wherein the transplantation rejection includes graft-versus-host disease (GVHD).

5. The method of claim 1, wherein the microbiome strain is selected from the group consisting of Lactobacillus rhamnosus, Lactobacillus paracasei, Lactobacillus gasseri, and Lactobacillus acidophilus.

6. The method of claim 1, wherein the composition increases probability of survival of graft.

7. The method of claim 1, wherein the composition regulates immune cells.

8. The method of claim 7, wherein the immune cells are cells selected from the group consisting of Th1, Th17, Treg, IL-10 producing B cell, plasma B cell, alloresponse T cell, Breg, B10, and CD19+IL-10+cell.

9. The method of claim 7, wherein the regulating of the immune cells suppresses activity of Th1, Th17, plasma B cell, or alloresponse T cell.

10. The method of claim 7, wherein the regulating of the immune cells increases the expression of immunoregulatory cells reduced by treatment of the immunosuppressant.

11. The method of claim 7, wherein the regulating of the immune cells increases the activity of cells selected from the group consisting of Treg, IL-10 producing B cell, Breg, B10 and CD19+IL-10+cell.

12. The method of claim 1, wherein the composition protects tissue damage.

13. The method of claim 12, wherein the tissue is a tissue selected from the group consisting of bone marrow, heart, cornea, intestine, liver, lung, pancreas, kidney and skin.

14. The method of claim 1, wherein the composition decreases the expression of IL-17.

15. The method of claim 1, wherein the composition increases the expression of IL-10 or IFN-γ.

16. A method for post-transplant immunosuppression, comprising administering to a subject in need thereof a composition comprising the compound represented by Formula 1 below or a pharmaceutically acceptable salt thereof and a microbiome strain as active ingredients
[Formula 1] [chemical structure]
